(19) United States
(12) Patent Application Publication
(10) Pub. No.: US 2018/0080002 A1
(43) Pub. Date: Mar. 22, 2018

(54) CELL DISPERSION DEVICE, AND AUTOMATIC SUBCULTURE SYSTEM USING SAME

(71) Applicant: HITACHI HIGH-TECHNOLOGIES CORPORATION, Minato-ku, Tokyo (JP)

(72) Inventors: Akihiro SHIMASE, Tokyo (JP); Kazumichi IMAI, Tokyo (JP); Sadamitsu ASO, Tokyo (JP); Eiichiro TAKADA, Tokyo (JP); Masako KAWARAI, Tokyo (JP); Toshinari SAKURAI, Tokyo (JP)

(21) Appl. No.: 15/326,938

(22) PCT Filed: Jul. 8, 2015
(86) PCT No.: PCT/JP2015/069585

§ 371 Date: Jan. 17, 2017

(30) Foreign Application Priority Data
Jul. 22, 2014 (JP) 2014-148762

(51) Int. Cl. C12M 1/36; C12M 1/00; C12M 1/34; C12M 1/33

(52) U.S. Cl. CPC C12M 41/48; C12M 45/02; C12M 41/00; C12M 29/00

(57) ABSTRACT

The purpose of the present invention is to provide a means for dispersing cell aggregates without damaging the cells, such that a sufficient multiplication rate can be obtained in a subculture. According to the present invention, provided is a cell-suspension processing device which disperses cell aggregates included in a cell suspension. The device is provided with: an inlet for taking in the cell suspension; an outlet for discharging the processed cell suspension; and a flow path which is provided between the inlet and the outlet, and which is capable of holding the cell suspension. The flow path has, provided thereto, a liquid delivery pump for causing the cell suspension inside to flow, a cell-dispersion-degree measurement instrument for measuring the dispersion degree of cells in the cell suspension, and a narrow part for imparting shearing force to the cell suspension flowing inside. The cell-suspension processing device is further provided with a control unit for controlling at least the liquid delivery pump on the basis of data obtained by the cell-dispersion-degree measurement instrument. The control unit determines whether the cells have attained a prescribed dispersion degree on the basis of the data obtained by the cell-dispersion-degree measurement instrument, and, in cases when the cells have not attained the prescribed dispersion degree, drives the liquid delivery pump such that the cell suspension is passed through the narrow part.

### TECHNICAL FIELD

The present invention relates to a device which cultures cells automatically and especially relates to a device which can conduct the subculture operation automatically.

### BACKGROUND ART

When anchorage-dependent cells are cultured in a container to which the cells are adhered, such as a petri dish or a flask, most of the operations have been conducted manually. Because the cell culture operations are complex and take a long time, enormous labor costs are required. Also, because the timings of the medium exchange and the subculture operation and the like depend on the experiences of the operators, a difference in the viability arises due to a difference in the degree to which cells are damaged, and the state of cells after the subculture operation is apt to vary with the operators. Thus, devices for automating the cell culture operations have been studied and developed so that cells can be cultured stably at low costs.

For example, PTL 1 proposes a cell culture device which automates the recovery of cultured cells and which can subculture the cells efficiently. Moreover, PTL 2 proposes that the risk of contamination during the culture operations is decreased using a cell culture device having culture dishes for culturing cells and control means for selectively transporting a cell solution to a prescribed culture dish.

### CITATION LIST

### Patent Literature

PTL 1: JP-A-2008-079554

PTL 2: JP-A-2007-185165

### SUMMARY OF INVENTION

### Technical Problem

In subculturing, operations of recovering cells after extended culturing and seeding the cells again after diluting the cells to an adequate cell concentration are required. However, in a cell suspension recovered from an extended culture device, cells often form aggregates and are rarely dispersed fully. When cells in such a state are seeded again, a sufficient multiplication rate cannot be obtained in subculturing. To increase the multiplication rate, it is necessary to separate the aggregates so that cells are separated and dispersed. Methods for dispersing cell aggregates include a method using an enzyme such as trypsin, a method for mechanically dispersing cell aggregates such as pipetting and the like. However, all of the methods damage cells when the dispersion is excessive and cause problems such as a decrease in the viability during subculturing. Therefore, especially in a cell culture device which subcultures cells automatically, means for dispersing cell aggregates without damaging the cells, such that a sufficient multiplication rate can be obtained in a subculture is desired.

### Solution to Problem

The invention provides a device which can disperse cells by imparting shearing force to cell aggregates at an adequate strength while confirming the degree of dispersion of the cells. The gist of the invention is as follows.

(1) A cell-suspension processing device for dispersing cell aggregates included in a cell suspension,

having an inlet for taking in the cell suspension, an outlet for discharging the processed cell suspension and a flow path which is provided between the inlet and the outlet and which is capable of holding the cell suspension,

wherein the flow path has, provided thereto, a liquid delivery pump for causing the cell suspension inside to flow, a cell-dispersion-degree measurement instrument for measuring the dispersion degree of cells in the cell suspension and a narrow part for imparting shearing force to the cell suspension flowing inside,

the cell-suspension processing device has a control unit for controlling at least the liquid delivery pump on the basis of data obtained by the cell-dispersion-degree measurement instrument, and

the control unit determines whether the cells have attained a prescribed dispersion degree on the basis of the data obtained by the cell-dispersion-degree measurement instrument and, in a case when the cells have not attained the prescribed dispersion degree, drives the liquid delivery pump such that the cell suspension is passed through the narrow part.

(2) The cell-suspension processing device according to (1), wherein the narrow part is provided by a flow-path pressing mechanism, which presses a flow path made of an elastic material and which sets the degree of narrowness of the flow path at any degree, and the control unit controls the flow-path pressing mechanism on the basis of the data obtained by the cell-dispersion-degree measurement instrument.

(3) The cell-suspension processing device according to (1), wherein the flow path has a parallel flow path part in which at least two or more flow paths are provided in parallel and which is designed such that a part of the flow paths is selected with a switch valve to pass the cell suspension, and the narrow part is provided in at least one of the flow paths included in the parallel flow path part.

(4) The cell-suspension processing device according to (3), wherein narrow parts are provided in two or more of the flow paths included in the parallel flow path part, and the sectional areas of the narrow parts are different.

(5) The cell-suspension processing device according to (3) or (4), wherein the control unit is capable of controlling the switch valve, and the control unit controls the switch valve such that any of the flow paths in the parallel flow path part is selected on the basis of the data obtained by the cell-dispersion-degree measurement instrument.

(6) The cell-suspension processing device according to any one of (1) to (4), wherein the cell-dispersion-degree measurement instrument measures the intensity of the scattered light or the transmitted light of light applied to the cell suspension and collects data on the dispersion degree of cells as a light intensity value, and the control unit determines the degree of dispersion of the cell aggregates on the basis of the change with time in the light intensity value.

(7) An automatic subculture system including a first cell culture device for extended culturing, a cell-suspension processing device for dispersing cell aggregates included in a cell suspension and a second cell culture device for subculturing,

wherein the cell-suspension processing device has an inlet for taking in a cell suspension discharged from the first cell culture device, an outlet for discharging the processed cell suspension and a flow path which is provided between the inlet and the outlet and which is capable of holding the cell suspension,

the flow path has, provided thereto, a liquid delivery pump for causing the cell suspension inside to flow, a cell-dispersion-degree measurement instrument for measuring the dispersion degree of cells in the cell suspension and a narrow part for imparting shearing force to the cell suspension flowing inside,

the cell-suspension processing device has a control unit for controlling at least the liquid delivery pump on the basis of data obtained by the cell-dispersion-degree measurement instrument, and

the control unit determines whether the cells have attained a prescribed dispersion degree on the basis of the data obtained by the cell-dispersion-degree measurement instrument and, in a case when the ceils have not attained the prescribed dispersion degree, drives the liquid delivery pump such that the cell suspension is passed through the narrow part.

The invention further includes the following inventions.

(1) A cell-number adjusting device, having

an inlet for taking in a cell suspension containing cells at a high concentration,

an outlet for discharging a cell suspension containing cells at a desired concentration which is lower than the concentration at the inlet, and

a flow path which is capable of holding a cell suspension between the inlet and the outlet,

characterized in that the flow path has, provided thereto, a liquid delivery pump for causing the cell suspension inside to flow, a cell counter for collecting data on the cell concentration per unit amount of the cell suspension and a diluent container for holding a diluent which is supplied to the flow path to dilute the cell suspension,

the cell-number adjusting device further has a control unit for controlling at least the liquid delivery pump on the basis of the data obtained by the cell counter, and

the control unit determines the amount of the diluent necessary for adjusting the cell concentration at the desired concentration on the basis of the data obtained by the cell counter, takes the necessary amount of the diluent into the flow path and drives the liquid delivery pump such that the cell suspension and the diluent are mixed.

(2) The cell-number adjusting device according to (1), wherein at least a part of the flow path provided between the inlet and the outlet forms a circulation flow path, the liquid delivery pump and the cell counter are provided in the circulation flow path, and the control unit drives the liquid delivery pump until the change in the data obtained from the cell counter falls in a predetermined value range and causes the cell suspension and the diluent to flow repeatedly in the circulation flow path to mix the cell suspension and the diluent.

(3) The cell-number adjusting device according to (2), further having a buffer tank in the circulation flow path.

(4) The cell-number adjusting device according to (1), wherein the control unit drives the liquid delivery pump alternately in the forward direction and in the backward direction to mix the cell suspension and the diluent.

(5) The cell-number adjusting device according to any one of (1) to (4), wherein the cell counter measures the intensity of the scattered light or the transmitted light of light applied to the cell suspension and collects data on the cell concentration as a light intensity value, and the control unit calculates the cell concentration by comparing the data with a relation between the cell concentration and the light intensity value determined in advance.

(6) The cell-number adjusting device according to any one of (1) to (4), wherein the cell counter collects the data on the cell concentration intermittently or continuously while the cell suspension is kept flowing.

(7) The cell-number adjusting device according to any one of (1) to (4), wherein the control unit is capable of controlling a valve which controls the intake of the cell suspension from the inlet and a valve which controls the intake of the diluent into the flow path, and the control unit controls the liquid delivery pump and the two valves such that the cell suspension and the diluent are taken in alternately and repeatedly.

(8) An automatic subculture system including a first cell culture device for extended culturing, a cell-number adjusting-device and a second cell culture device for subculturing,

wherein the first cell culture device discharges a cell suspension having a high concentration, the cell-number adjusting device dilutes the cell suspension having a high concentration to obtain a uniform cell suspension having a desired cell concentration, the second cell culture device seeds the diluted cell suspension and subcultures the cells,

the cell-number adjusting device has

an inlet for taking in the cell suspension having a high concentration,

an outlet for discharging a cell suspension containing cells at a desired concentration which is lower than the concentration at the inlet,

a flow path which is capable of holding a cell suspension between the inlet and the outlet,

the flow path has, provided thereto, a cell counter for collecting data on the cell concentration per unit amount of the cell suspension and a diluent container for holding a diluent which is supplied to the flow path to dilute the cell suspension,

the cell-number adjusting device further has a control unit for controlling the flow of the cell suspension in the flow path on the basis of the data obtained by the cell counter, and

the control unit determines the amount of the diluent necessary for adjusting the cell concentration at the desired concentration on the basis of the data obtained by the cell counter, takes the necessary amount of the diluent into the flow path and controls the flow of the cell suspension in the flow path such that the cell suspension and the diluent are mixed.

(9) The automatic subculture system according to (8), wherein the control unit controls the flow of the cell suspension in the flow path of the cell-number adjusting device using a liquid delivery pump provided in the first cell culture device or the second cell culture device.

(10) A method for diluting a cell suspension containing cells at a high concentration to a desired concentration, including

a step of measuring the intensity of the scattered light or the transmitted light of light applied to the cell suspension intermittently or continuously while keeping the cell suspension flowing and collecting data on the cell concentration as a light intensity value,

a step of converting the obtained data into a cell concentration by comparing the data with a relation between the cell concentration and the light intensity value determined in advance, and

a step of calculating the amount of a diluent necessary for diluting the cell suspension to the desired concentration and adding and mixing the amount of the diluent with the cell suspension.

### Advantageous Effects of Invention

According to the invention, cell aggregates contained in a cell suspension obtained by extended culturing can be dispersed at an adequate strength regardless of the level of skill of the operator, and a stable subculture operation becomes possible. The invention contributes to the achievement of stable cell culturing on the sites of regenerative medicine and the like.

This description includes the contents described in the description, the claims and the drawings of patent application No. 2014-148762 to which this application claims priority.

### BRIEF DESCRIPTION OF DRAWINGS

FIG. 1 A schematic figure showing the first embodiment of the cell dispersion device of the invention.

FIG. 2 A conceptual figure showing the change with time in the light intensity value caused when cell aggregates are dispersed by switching the direction of rotation of the peristaltic pump 4.

FIG. 3 A schematic figure showing the second embodiment of the cell dispersion device of the invention.

FIG. 4 A schematic figure showing the third embodiment of the cell dispersion device of the invention.

FIG. 5 A conceptual figure showing the change with time in the light intensity value caused when a cell suspension containing cell aggregates is passed through the cell dispersion device of the third embodiment.

FIG. 6 A schematic figure showing the fourth embodiment of the cell dispersion device of the invention.

FIG. 7 A schematic FIG. of the structures of the flow-path pressing mechanism 9. The figures on the left show the side of a flow path, and the figures on the right show the section of a flow path.

FIG. 8 A schematic figure showing the fifth embodiment of the cell dispersion device of the invention.

FIG. 9 A schematic figure showing the whole structure of the subculture system of the invention.

FIG. 10 A schematic figure showing the whole structure of a subculture system using an open-system cell culture device.

FIG. 11 A schematic figure showing the first embodiment of the cell dispersion device having a cell-number adjusting function of the invention.

FIG. 12 A schematic figure showing the second embodiment of the cell dispersion device having a cell-number adjusting function of the invention.

FIG. 13 A schematic figure showing the third embodiment of the cell dispersion device having a cell-number adjusting function of the invention.

FIG. 14 A schematic figure showing the cell dispersion device having a cell-number adjusting function according to a variation of the third embodiment.

FIG. 15 A conceptual figure showing the change with time in the light intensity value which is output from the detector 7 when a cell suspension is passed through the cell dispersion device 122 having a cell-number adjusting function according to the third embodiment or the variation 123 thereof.

FIG. 16 A schematic figure showing the whole structure of a subculture system using a cell dispersion device having a cell-number adjusting function.

FIG. 17 A schematic figure showing a part of the structure of the first variation of a subculture system using a cell dispersion device having a cell-number adjusting function.

FIG. 18 A schematic figure showing a part of the structure of the second variation of a subculture system using a cell dispersion device having a cell-number adjusting function.

FIG. 19 A schematic figure showing a part of the structure of the third variation of a subculture system using a cell dispersion device having a cell-number adjusting function.

FIG. 20 A schematic figure showing a part of the structure of the fourth variation of a subculture system using a cell dispersion device having a cell-number adjusting function.

FIG. 21 A schematic figure showing a part of the structure of the fifth variation of a subculture system using a cell dispersion device having a cell-number adjusting function.

FIG. 22 A schematic figure showing the whole structure of an open subculture system using a cell dispersion device having a cell-number adjusting function.

FIG. 23 A graph showing how the values changed when the scattered light intensities were measured intermittently with a detector provided in a flow cell of a cell dispersion device to which a cell suspension was supplied.

FIG. 24 A schematic FIG. of the cell dispersion device used for automatic optimization of the conditions for dispersing cells.

### DESCRIPTION OF EMBODIMENTS

### Cell Dispersion Device: First Embodiment

FIG. 1 is a schematic figure showing the first embodiment of the cell dispersion device of the invention. The cell dispersion device 110 according to the first embodiment has a function of taking in a cell suspension having an unknown degree of dispersion of cells from an inlet 1, dispersing cell aggregates inside and discharging a cell suspension in which the cells are uniformly dispersed from an outlet 2. The inlet 1 and the outlet 2 are connected with a flow path 3, and a peristaltic pump 4 which is a liquid delivery pump for causing a liquid in the flow path to flow is provided. A control unit 11 controls at least the peristaltic pump 4. The flow path 3 does not have to have a uniform tube diameter. The flow path 3 has a sufficient capacity for holding the cell suspension including the space for its movement.

At least a part of the flow path 3 has a part made of an elastic material, and the peristaltic pump 4 causes a fluid in the flow path to flow by squeezing the elastic part of the flow path 3. A peristaltic pump is preferable because the driving part such as a blade does not touch the fluid directly, thereby causing the fluid to flow without contamination, and because the damage to the dispersed cells is small. The pump for causing the fluid to flow is not limited to a peristaltic pump, but a pump whose driving part does not touch the fluid directly, like a peristaltic pump, is preferable. Such pumps are a diaphragm pump, a syringe pump and the like.

An orifice 8 which forms a flow path narrow part is inserted in the flow path 3. By changing the sectional area of the flow path suddenly with the orifice 8 and thus imparting strong shearing force to a fluid passing through the orifice 8, the dispersion of cell aggregates is promoted. It is preferable to pass the cell suspension through the orifice 8 repeatedly by repeatedly switching the direction of rotation of the peristaltic pump 4, because the cell aggregates are dispersed more easily. Considering that the sizes of cells are generally about 10 μm, the diameter (sectional diameter) of the orifice 8 is preferably in the range of 0.5 mm to 1 mm because the cell aggregates can be dispersed efficiently. Also, the diameter of the orifice may be changed to a value suitable for each cell kind on the basis of the cell size and the adhesiveness. An inexpensive orifice made of resin is preferably used for the orifice 8, because the flow path including the orifice 8 can be made disposable if necessary.

A flow cell 5 is provided in a part of the flow path 3, and the light intensity is measured as data on the degree of dispersion of the cell aggregates when the cell suspension passes through the flow cell 5. Light from a light source 6 is applied to the flow cell 5, and the transmitted light or the scattered light thereof, or both thereof, is detected by a detector 7. In this embodiment, the light source 6 and the detector 7 compose a cell-dispersion-degree measurement instrument.

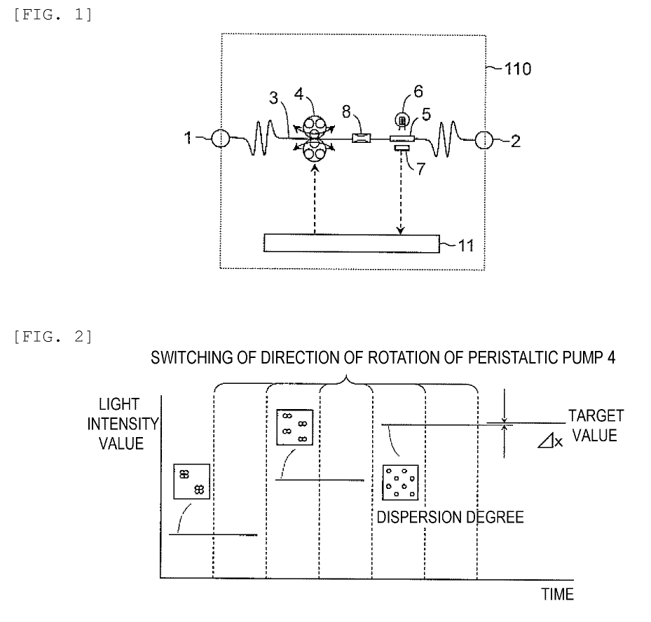

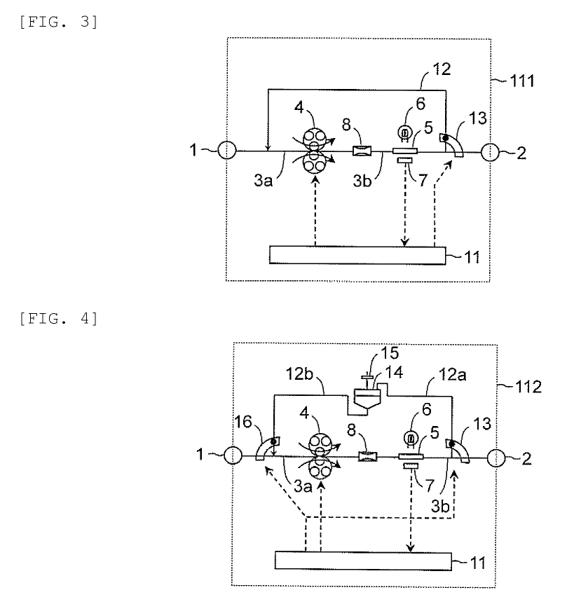

The quantity of the transmitted light or the scattered light observed from the flow cell 5 changes as the dispersion degree of the cells in the cell suspension changes. Thus, focusing on the change with time in the light intensity detected by the detector 7, it becomes possible to determine that cells are fully dispersed, as the variation in the light intensity values becomes small and the light intensity values converge on a certain value (preferably a predetermined target value). The control unit 11 determines whether the cells have attained a prescribed dispersion degree on the basis of the light intensity data obtained by the detector 7 and, in a case when the cells have not attained the prescribed dispersion degree, drives the peristaltic pump 4 such that the cell suspension passes through the orifice 8. For example, by switching the direction of rotation of the peristaltic pump 4, the cell suspension is caused to pass through the orifice 8 repeatedly. When the peristaltic pump 4 is driven in this manner, shearing force is imparted to the cell suspension also at a part other than the orifice 8, and an effect of dispersing the cell aggregates or uniformly stirring the cell suspension in the flow path is also obtained. Also by changing the liquid delivery speed of the peristaltic pump 4, shearing force can be imparted to the cell suspension. FIG. 2 is a conceptual figure showing the change with time in the light intensity value caused when cell aggregates are dispersed by switching the direction of rotation of the peristaltic pump 4.

It is particularly preferable to employ the method in which light from the light source 6 is applied to the flow cell 5 and the transmitted light or the scattered light thereof, or both thereof, is detected by the detector 7, as described above, as the method for measuring the dispersion degree of the cells, because the dispersion degree of the cells can be measured while the cell suspension is kept flowing. However, the method for measuring the dispersion degree of the cells is not limited to this method, and another method may be employed. For example, the dispersion degree of the cells may be calculated from an image by providing any observation window in the flow path 3 and taking an image (a still image or a video) with a microscope equipped with a CCD camera. To measure the dispersion degree of the cells while the cell suspension is kept flowing, real-time processing is required. Means which is capable of conducting such rapid image processing can be employed as the means for measuring the dispersion degree of the cells instead of the light intensity measurement.

A material which does not affect the cells or which affects the cells very little is preferably used for the tube composing the flow path 3. An example of such a material is a silicone tube for medical use. Also, although the flow cell 5 may be made of glass, an inexpensive flow cell made of resin is more preferably used because it is easy to make the flow cell 5 through which cells have passed once, including the flow path 3, disposable.

### Cell Dispersion Device: Second Embodiment

FIG. 3 is a schematic figure showing the second embodiment of the cell dispersion device of the invention. The basic structure of the cell dispersion device 111 according to the second embodiment is similar to that of the first embodiment, but a difference is that the flow path after the peristaltic pump 4 is branched and returned to the flow path before the peristaltic pump 4 so that the flow path has a circular structure. The flow path before the pump, the flow path after the pump and the branched feedback flow path are indicated by 3a, 3b and 12, respectively. A switch valve 13 is provided at the branched part to the feedback flow path 12 so that the flow path at the outlet 2 side and the feedback flow path 12 can be selected. With such a structure, the cell suspension can be passed through the orifice 8 repeatedly even without switching the direction of rotation of the peristaltic pump 4, and effects such as improvement of the stability of the measurement of the dispersion degree of the cells through the measurement of the light intensity or the like, reduction in the load to the peristaltic pump 4, simplification of the control with the control unit 11 and reduction in the load to the cells are obtained.

At the point where the feedback flow path 12 joins, the pressure in the flow path 3a at the pump side is lower than the pressure at the inlet 1 side, and thus the liquid flowing from the feedback flow path 12 flows to the pump side and does not flow backward to the inlet 1 side. However, because the amount of backflow is not absolute zero, a pinch valve or a nonreturn valve for preventing backflow may be provided in the flow path 3a at a point which is closer to the inlet 1 than the point where the feedback flow path 12 joins.

### Cell Dispersion Device: Third Embodiment

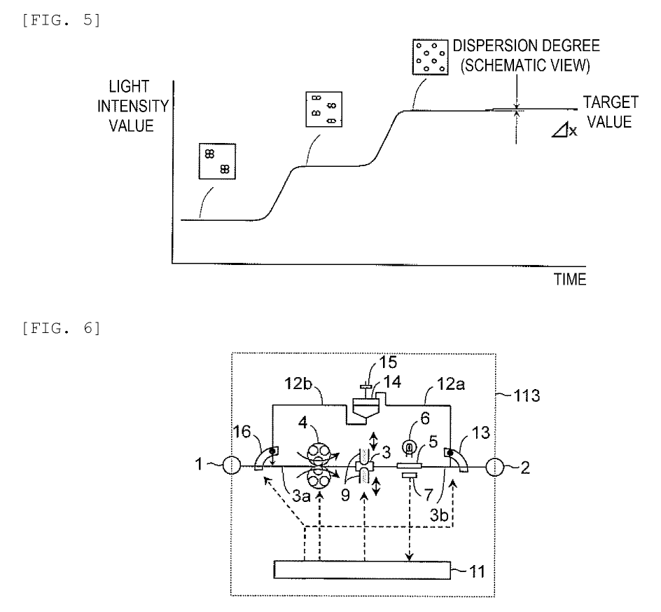

FIG. 4 is a schematic figure showing the third embodiment of the cell dispersion device of the invention. The basic structure of the cell dispersion device 112 according to the third embodiment is similar to that of the second embodiment, but a difference is that a buffer tank 14 is provided in the feedback flow path 12.

A structure having a circulation flow path as in the second embodiment is advantageous in controlling the cell dispersion. However, there is a restriction because the cells have to be dispersed within the capacity of the circulation flow path. The volume of the cell suspension taken in from the inlet 1 is unknown, and the total volume of the liquid to be held in the circulation flow path can vary. Although the length of the circulation flow path may be made long so that the capacity of the circulation flow path can correspond to the maximum expected liquid volume, it is believed that the efficiency of the cell dispersion decreases when the actual liquid volume is less than the maximum liquid volume. In the third embodiment shown in FIG. 4, this problem is solved by providing the buffer tank 14 and thereby changing the circulation capacity.

The buffer tank 14 is provided in the middle of the feedback flow path 12, and the flow paths before and after the buffer tank are indicated by 12a and 12b, respectively. For example, 12a and 12b are connected to the buffer tank 14 in such a way that 12a enters from the top of the buffer tank and 12b exits from the bottom of the tank. The buffer tank 14 may be open to the atmosphere, and in this case, it is preferable to provide a HEPA filter 15 in the middle to prevent the contamination by germs from outside. A switch valve 16 is provided at the point where the feedback flow path 12b joins so that the flow path at the inlet 1 side and the flow path at the peristaltic pump side can be selected. Use of a universal switch valve with which the two flow paths can be opened and closed simultaneously and alternately with one actuator is preferable when the control unit 11 controls the switch valve 16. When a cell suspension containing cell aggregates is passed through the cell dispersion device of the third embodiment, the light intensity value output from the detector 7 changes with time as shown in FIG. 5.

The purpose of the buffer tank is to make the liquid volume to be handled variable, and the tank does not have to have the structure shown in the figure. For example, a liquid bag made of a stretch material or a bag which has a paper-folding structure and which is folded to change the capacity freely may be used as the buffer tank. Such a bag may have a structure for releasing air incorporated therein or may have a structure for trapping air in the bag rather than releasing the air. A liquid only can be discharged without mixing air in when the outlet of the bag is provided at the bottom.

### Cell Dispersion Device: Fourth Embodiment

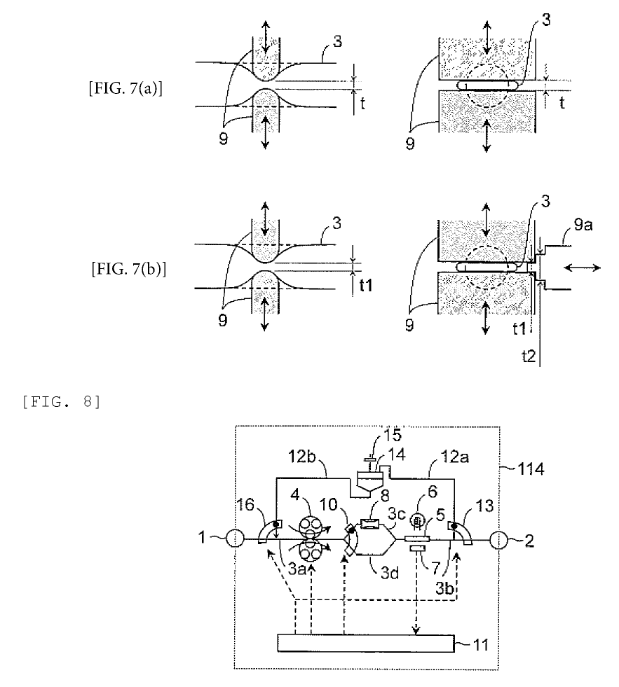

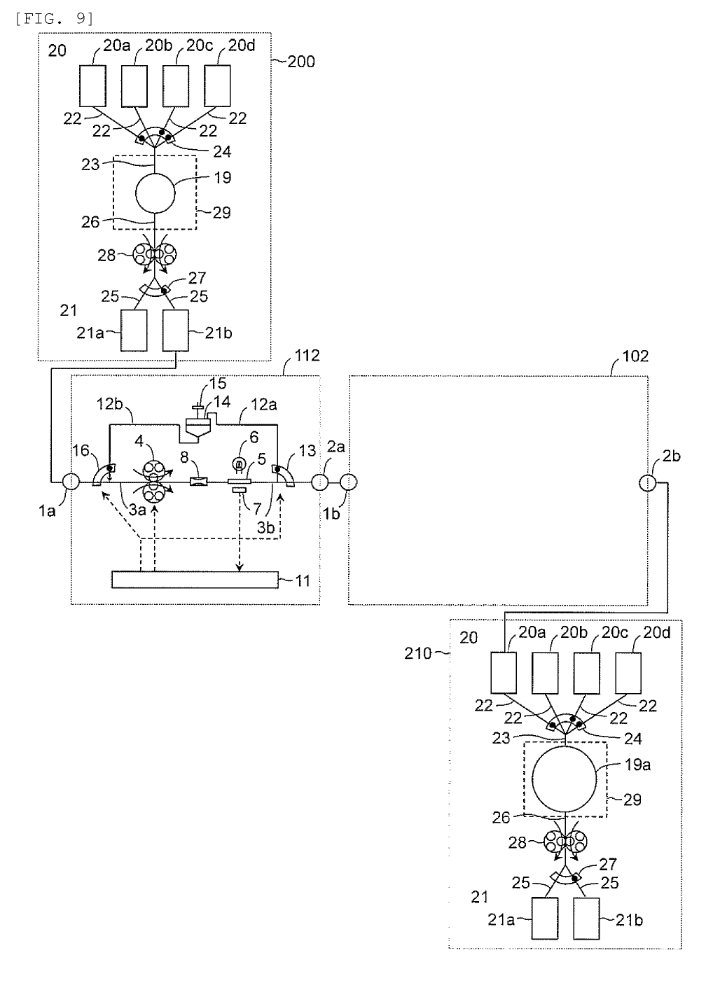

FIG. 6 is a schematic figure showing the fourth embodiment of the cell dispersion device of the invention. The cell dispersion device 113 according to the fourth embodiment is characterized by having a flow-path pressing mechanism 9 which can control the degree of pressing of the flow path instead of the orifice 8. FIG. 7 is a schematic figure of the structures of the flow-path pressing mechanism 9. The flow-path pressing mechanism 9 has a function of pressing an elastic flow path from outside and presses the flow path while keeping a certain space, rather than completely closing as a pinch valve. The flow-path pressing mechanism 9 is preferably controlled by the control unit 11. By changing the degree of pressing of the flow path, the shearing force imparted to the cell aggregates in the cell suspension flowing inside can be changed. Also, in the case where the cell aggregates are still large, the flow path may be clogged with the cells when the sectional area of the narrow part of the flow path is too small. However, when the flow-path pressing mechanism 9, which can change the degree of pressing of the flow path, is used, such a problem can be avoided by selecting an adequate degree of pressing of the flow path.

The control unit 11 preferably controls the flow-path pressing mechanism 9 on the basis of data on the dispersion degree of the cells obtained from the cell-dispersion-degree measurement instrument and changes the degree of pressing of the flow path. For example, the flow-path pressing mechanism 9 may be able to change the space t from the fully open state in which the flow path is not pressed at all to the closed state in which the flow path is completely pressed as shown in FIG. 7(a) and control the size of the space t using an actuator which can determine the position like a stepper motor. Alternatively, as shown in FIG. 7(b), the space t may be determined by inserting a member 9a which serves as an indicator of the space size. Such member 9a may be able to correspond to more than one space size. For example, the member 9a shown in FIG. 7(b) can correspond to the space sizes t1 and t2 and full open. In this regard, the flow-path pressing mechanism 9 may be employed instead of the orifice 8 also in the cell dispersion devices 110 and 111 explained using FIGS. 1 and 2.

### Cell Dispersion Device: Fifth Embodiment

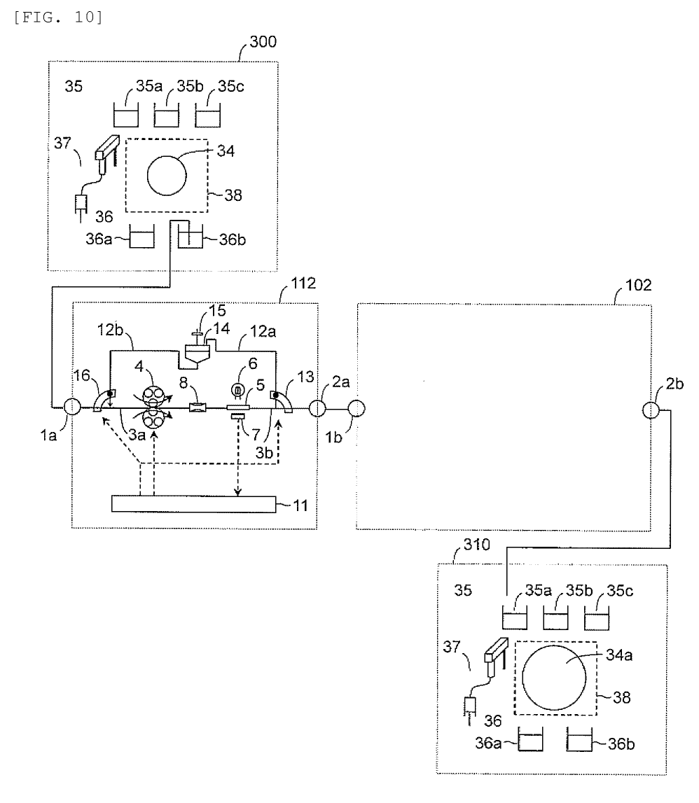

FIG. 8 is a schematic figure showing the fifth embodiment of the cell dispersion device of the invention. The cell dispersion device 114 according to the fifth embodiment is characterized by having a parallel flow path part in which a flow path 3c having the orifice 8 and a flow path 3d having no orifice are connected in parallel and can be each selected with a switch valve 10. Not only one but also two or more flow paths 3c having the orifice 8 may be prepared, and the orifices may nave diameters different from each other. In this manner, on the basis of the data on the dispersion degree of the cells obtained from the cell-dispersion-degree measurement instrument, the cell aggregates can be caused to pass through an orifice having a large diameter for example when the cell aggregates are determined to be relatively large, and the cell aggregates can be caused to pass through a smaller orifice when the cell aggregates are determined to have been separated to some extent. A flow path 3c can be selected by controlling the switch valve 10 with the control unit 11. With such a structure, appropriate cell dispersion treatment can be conducted on the basis of the data on the dispersion degree of the cells obtained from the cell-dispersion-degree measurement instrument without a complex structure like the flow-path pressing mechanism 9 explained using FIGS. 6 and 7, and the orifices 8 can be prevented from being clogged.

### (Closed Subculture System Using Cell Dispersion Device)

A subculture system using the cell dispersion device of the invention is explained below. Because the cell dispersion device of the invention forms a closed system without contamination, by germs from outside when the inlet and the outlet are closed, the whole system can be made a closed system when closed-system cell culture devices are connected. An example in which closed-system cell culture devices are connected is explained below.

FIG. 9 is a schematic figure showing the whole structure of the subculture system of the invention. In a closed-system cell culture device 200, a culture container 19 is connected to a supply bag 20 and a recovery bag 21, and thus one closed system is formed. By culturing cells in a closed system, cells can be cultured safely and reliably without contamination by germs from outside. More than one supply bag 20 may be used. Individual flow paths 22 connected to the respective bags are in parallel and are all connected to a common flow path 23, and any one of the supply bags 20 can be selected with a switch valve 24 provided on the individual flow paths 22. Here, it is supposed that the supply bags contain a cell suspension 20a, a medium 20b, a detachment solution 20c and sterilized air 20d, but the contents of the supply bags are not limited to these examples. In this regard, the sterilized air is used for pushing out a liquid which is already contained from behind and discharging the liquid. A HEPA filter may be connected instead of the supply bags to make the system open to the atmosphere. The HEPA filter can prevent contamination by germs.

Similarly, more than one recovery bag 21 may be used. Individual flow paths 25 are in parallel and are all connected to a common flow path 26, and any one of the recovery bags 21 can be selected with a switch valve 27 provided on the individual flow paths 25. Here, it is supposed that the recovery bags contain a liquid waste 21a and a cell suspension 21b, but the contents of the recovery bags 21 are not limited to these examples.

By selecting any one of the supply bags 20 and any one of the recovery bags 21 with the switch valves 24 and 27 and driving a peristaltic pump 28, a liquid necessary for culturing is delivered. After seeding the cell suspension 20a in the culture container 19, the medium is changed regularly, and cells are cultured. After culturing cells, the cells are detached from the culture container 19 using the detachment solution 20c and recovered in the recovery bag 21b.

In the following manner, the closed-system cell culture devices and the cell dispersion device are connected, and passage of cells is conducted. There are two culture devices, namely the closed-system cell culture device 200 for the first extended culturing and a closed-system cell culture device 210 for culturing after passage. The basic structures of the two culture devices are the same. Because the culture volume of the latter is larger, a container having a larger area may be used for the latter, or using more than one culture container connected in parallel, a liquid may be delivered while the culture containers are switched with a switch valve which is not shown in the figure.

The recovery bag 21b containing a cell suspension of the culture device 200 and the inlet 1a of the cell dispersion device 112, the outlet 2a of the cell dispersion device 112 and an inlet 1b of a cell-number adjusting device 102, and an outlet 2b of the cell-number adjusting device 102 and a supply bag 20a of the culture device 210 to which a cell suspension is supplied are each connected with a connecting flow path. The cell-number adjusting device 102 is a device having a function of taking in a cell number suspension having an unknown cell concentration and discharging a uniform cell number suspension which has been diluted to a desired concentration, and the cell-number adjusting device 102 may have any structure as long as the function can be achieved. Also, for example, the cell-number adjusting device 102 can be omitted by further providing a branch flow path and a diluent bag connected thereto in the flow path of the cell dispersion device 112 and adding a component which determines the concentration of the cell suspension on the basis of the light intensity data detected by the detector 7 composing the cell-dispersion-degree measurement instrument and which takes in a necessary amount of a diluent from the diluent bag. The cell dispersion device 112 used in this system is the cell dispersion device according to the third embodiment described above, but the cell dispersion device according to another embodiment may also be used.

The cell dispersion device 112 drives the peristaltic pump 4 and takes in the cell suspension from the recovery bag 21b. Because the amount of the cell suspension can change depending on the results of extended culturing and the like, it is preferable to drive the peristaltic pump 4 for a long time and to deliver the total amount once to the buffer tank 14. The switch valve 16 is provided in the cell dispersion device 112 at the point where the feedback flow path 12b joins so that the flow path at the inlet 1a side and the flow path at the peristaltic pump side can be selected. A universal switch valve with which the two flow paths can be closed and opened simultaneously and controlled alternately with one actuator is preferably used. Next, by circulating the cell suspension in the circulation flow path composed of the feedback flow paths 12a and 12b including the buffer tank 14 and the flow paths 3a and 3b after switching the switch valve 16 such that the feedback flow path 12b is selected, the cell suspension is passed through the orifice 8 repeatedly, and shearing force is imparted to the cell aggregates to disperse the cell aggregates. The cell-dispersion-degree measurement instrument composed of the light source 6 and the detector 7 detects the transmitted light or the scattered light, or both thereof, in the flow cell 5 by the detector 7 and outputs the results to the control unit 11. The control unit 11 determines the degree of dispersion of the cell aggregates on the basis of the light intensity value. After dispersing the cell aggregates, the cell suspension is delivered to the cell-number adjusting device 102 for example by controlling the switch valve 13, and the cell concentration is adjusted. Then, the cell suspension is delivered to the supply bag 20a for the cell suspension of the cell culture device 210. In the cell culture device 210 to which the cell suspension is supplied, cells are cultured in a similar manner to that in the culture device 200.

### (Open Subculture System Using Cell Dispersion Device)

A subculture system has been explained above assuming that closed-system cell culture devices are used. However, the subculture system of the invention is not limited to a closed system, but open-system cell culture devices can also be used. The open-system cell culture device is a device which cultures cells in an unsealed culture container, for example the medium is changed after the cover of the culture container is removed, as in a general cell culture method. Although the risk of contamination by germs is higher, an advantage is that a liquid can be handled more readily. The risk can be reduced by installing the device in a clean room.

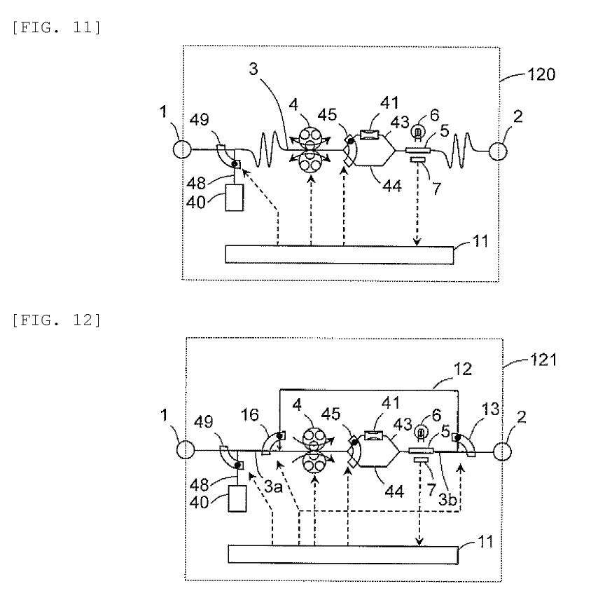

FIG. 10 is a schematic figure showing the whole structure of a subculture system using open-system cell culture devices. An open-system cell culture device 300 has an unsealed culture container 34, a supplied solution container 35 which is not sealed, either and a recovered solution container 36. Supplied solutions are a cell suspension 35a, a medium 35b and a detachment solution 35c, and recovered solutions are a liquid waste 36a and a cell suspension 36b. These liquids are sucked and discharged by a dispensing mechanism 37. The culture container is provided in an incubator 38, and cells are cultured in an environment suitable for culturing.

An inlet flow path and an output flow path are connected to the inlet 1a and the outlet 2a of the cell dispersion device 112, respectively, and the inlet flow path extends to a recovered liquid bottle 36b of the cell culture device 310 for extended culturing. The outlet flow path extends to a supplied liquid bottle 35a of a cell culture device 310 for subculturing through the cell-number adjusting device 102. The cell suspension which has been cultured and recovered in the cell culture device 300 for extended culturing is recovered in the recovered solution container 36b by the dispensing mechanism 37. The cell dispersion device 112 takes in the cell suspension from the inlet flow path, disperses cell aggregates and discharges the cell suspension to the cell-number adjusting-device 102. The cell-number adjusting device 102 takes in the cell suspension from an inlet flow path, adjusts the cell concentration and then discharges the cell suspension to the supplied solution container 35a of the cell culture device 310 for subculturing from an outlet flow path. In this manner, also when open-system cell culture devices are connected, the cell dispersion device 112 can be used in a similar manner to that when closed-system cell culture devices are connected.

### Cell Dispersion Device Having Cell-Number Adjusting Function: First Embodiment

In the subculture systems shown in FIG. 9 and FIG. 10, the cell-number adjusting device 102 is connected after the cell dispersion device 112. The adjustment of the cell number is necessary for making the cell concentration constant when the cells are seeded again in the cell culture device 210 or 310 and culturing cells stably. Although the dispersion of the cells and the adjustment of the cell number are conducted by separate devices in the subculture systems shown in FIG. 9 and FIG. 10, a device which can conduct both simultaneously is explained below.

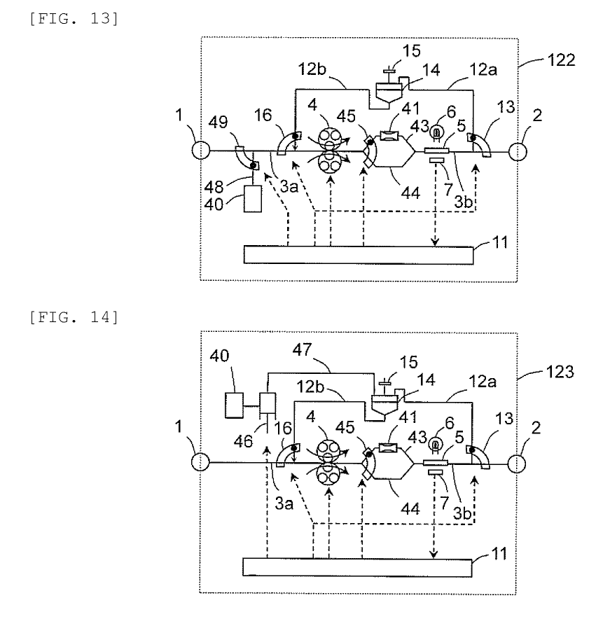

FIG. 11 is a schematic figure showing the first embodiment of the cell dispersion device having a cell-number adjusting function of the invention. The cell dispersion device 120 having a cell-number adjusting function according to the first embodiment has a similar structure to that of the cell dispersion device 110 shown in FIG. 1, but a difference is that a part of the flow path 3 is branched and connected to a branch flow path 48 and that a switch valve 49 is provided at the branched part. Moreover, as in the cell dispersion device 114 shown in FIG. 8, the cell dispersion device 120 having a cell-number adjusting function has a parallel flow path part in which a flow path 43 having an orifice 41 and a flow path 44 having no orifice are connected in parallel and can be each selected with a switch valve 45.

In the cell dispersion device 120 having a cell-number adjusting function, the light intensity measured by the detector 7 is also used as data on the cell concentration per unit amount. The relation between the intensity of the transmitted light or the scattered light detected by the detector 7 and the cell number is determined separately in advance, and the cell concentration is calculated on the basis of the relation and the light intensity detected by the detector 7. The relation between the intensity of the transmitted light or the scattered light and the cell number can be determined for example by preparing several kinds of cell suspensions having known concentrations of the cells to be cultured, measuring the light intensities of the cell suspensions and creating a calibration curve from the obtained results. In this regard, the flow amount of the cell suspension passing through the flow cell 5 can be determined on the basis of the amount taken in from the inlet 1 or on the basis of the capacity or the sectional area of the flow cell 5 and the liquid delivery speed of the peristaltic pump 4.

By the measurement of the cell concentration on the basis of the light intensity, the cell concentration can be calculated while the cell suspension is kept flowing. When the cell concentration is calculated while the cell suspension is kept flowing, the detector 7 may measure the light intensity continuously and incessantly or may measure the light intensity intermittently, namely at intervals, preferably at regular intervals. Another means may be used as the means for calculating the cell concentration.

The switch valve 49 provided in the branch flow path 48 can switch the branch flow path 48 and the flow path at the inlet 1 side. A pinch valve is preferably used for the switch valve. A pinch valve flattens (pinches) a flow path made of an elastic material from outside and controls a flow, and the pinch valve can control a fluid without contaminating the fluid or the valve itself because the pinch valve does not touch the fluid directly. The switch valve 49 has a function of switching the two flow paths and can be achieved by a combination of two pinch valves, but a universal type with which the two flow paths can be closed and opened simultaneously and controlled alternately with one actuator may also be used. The control unit 11 may control switching of the valve by controlling the actuator provided in the switch valve 49.

A diluent container 40 containing a diluent is connected to the end of the branch flow path 48. The control unit 11 controls at least the peristaltic pump 4, preferably the switch valve 49 as well, adds the diluent to the cell suspension taken in depending on the detection results of the detector 7 and thoroughly stirs the cell suspension and the added diluent so that the cell concentration becomes uniform. The control over the peristaltic pump 4, the switch valve 9 and the like by the control unit 11 is explained in detail below.

The control unit 11 drives the peristaltic pump 4 while the switch valve 49 blocks the branch flow path 48 and selects the flow path at the inlet 1 side, and the control unit 11 takes in the undiluted cell suspension from the inlet 1. The cell suspension taken in is directly transported to the flow cell 5. When the cell suspension passes through the flow cell 5, the light intensity is measured by the detector·BR>V. The control unit 11 calculates the cell concentration from the results of the measurement, compares with the predetermined target value and calculates and determines the necessary amount of the diluent in view of the amount of the undiluted solution taken in.

The control unit 11 then switches the switch valve 49 such that the branch flow path 48 side is selected, drives the peristaltic pump 4 for a certain period and takes in the diluent from the diluent container 40 into the flow path 3. In the flow path 3, two liquids, namely the cell suspension having a high cell concentration before adjustment and the diluent, exist ununiformly. Next, the control unit 11 mixes the two liquids by switching the direction of rotation of the peristaltic pump 4 to the normal rotation and the reverse rotation several times and moving the liquids forward and backward repeatedly in the flow path 3. The flow path 3 has a sufficient space for holding the cell suspension and the diluent including the space for the movement thereof. In this regard, the two liquids can be mixed not only by switching the direction of rotation of the peristaltic pump 4 but also by changing the flow rate for example by changing the speed of rotation of the peristaltic pump.

The measured values of the light intensity measurement vary widely in the beginning because the cell concentration in the flow path 3 is ununiform. However, as the direction of rotation of the peristaltic pump 4 is switched repeatedly, the cell concentration becomes gradually uniform, and the variation becomes small. In the end, the values converge on a target value, namely the light intensity value corresponding to the predetermined cell concentration. Thus, the control unit 11 determines that the liquid in the flow path 3 has become uniform at the point where the change with time in the measured values of the light intensity measurement falls within a prescribed value range (target value±Δx), preferably at the point where there is no change in the values anymore. If the limit of convergence differs from the target value, the control unit 11 may repeat the dilution step described above again. The cell suspension having a desired cell concentration after the dilution step is discharged from the outlet 2 by driving the peristaltic pump 4.

In the dilution step described above, the cell suspension is taken in from the inlet 1 once, and the diluent is taken in from the diluent container 40 once. However, the control unit 11 may switch the switch valve 49 within a shorter span and take in the cell suspension and the diluent alternately and repeatedly more than once in small divided portions. Such a manner is preferable because the two liquids are mixed more easily and because the load applied to the cells can be reduced.

As described above, in the cell dispersion device 120 having a cell-number adjusting function, the flow path 43 having the orifice 41 and the flow path 44 having no orifice are connected in parallel and can be each selected with the switch valve 45. As in the cell dispersion device 114 shown in FIG. 8, not only one but also two or more flow paths 41 having an orifice may be prepared, and the orifices may have diameters different from each other. On the basis of the data on the dispersion degree of the cells obtained from the cell-dispersion-degree measurement instrument, the cell aggregates can be caused to pass through an orifice having a large diameter for example when the cell aggregates are determined to be relatively large, and the cell aggregates can be caused to pass through a smaller orifice when the cell aggregates are determined to have been separated to some extent. Because shearing force is imparted when the liquids are passed through an orifice, an effect of promoting mixing of the cell suspension and the diluent is also obtained. Thus, a flow path 41 may be selected also depending on the mixing state with the diluent. In this regard, however, the parallel flow path part does not always have to be provided, and a structure having a single orifice in a single flow path as in the cell dispersion device 110 shown in FIG. 1 or a structure having a flow-path pressing mechanism as in the cell dispersion device 113 shown in FIG. 6 may also be used.

### Cell Dispersion Device Having Cell-Number Adjusting Function: Second Embodiment

FIG. 12 is a schematic figure showing the second embodiment of the cell dispersion device having a cell-number adjusting function of the invention. The basic structure of the cell dispersion device 121 having a cell-number adjusting function according to the second embodiment is similar to that of the first embodiment, but a difference is that the flow path after the peristaltic pump 4 is branched and returned to the flow path before the peristaltic pump 4 so that the flow path has a circular structure. The flow path before the pump, the flow path after the pump and the branched feedback flow path are indicated by 3a, 3b and 12, respectively. A switch valve 13 is provided at the branched part to the feedback flow path 12 so that the flow path at the outlet 2 side and the feedback flow path 12 can be selected. The branch flow path 48 to which the diluent container 40 is connected is provided at the flow path 3a side.

The dilution step by the cell dispersion device 121 having a cell-number adjusting function of the second embodiment is conducted as follows. First, the control unit 11 controls the switch valve 13 such that the feedback flow path 12 is blocked and that the flow path at the outlet 2 side is selected and controls the switch valve 49 such that the branch flow path 48 is blocked and that the flow path at the inlet 1 side is selected. In this state, the control unit 11 drives the peristaltic pump 4 and takes in the undiluted cell suspension from the inlet 1. The light intensity is measured and the diluent is taken in in similar manners to those in the first embodiment.

In the second embodiment, two liquids, namely the undiluted cell suspension having a high cell concentration before adjustment and the diluent, are mixed using the flow paths 3a and 3b and the feedback flow path 12. First, the control unit 11 switches the switch valve 13 such that the feedback flow path 12 is selected and drives the peristaltic pump 4 in this state. The cell suspension and the diluent are stirred while circulating in the circulation flow path composed of the feedback flow path 12 and the flow paths 3a and 3b, and the cell concentration becomes gradually uniform. According to the second embodiment, the liquids can be mixed without switching the direction of rotation of the peristaltic pump 4 because the feedback flow path 12 is provided, and effects such as improvement of the stability of the measurement of the cell concentration through the measurement of the light intensity or the like, reduction in the load to the peristaltic pump 4, simplification of the control with the control unit 11 and reduction in the load to the cells are obtained.

At the point where the feedback flow path 12 joins, the pressure in the flow path 3a at the pump side is lower than the pressure at the inlet 1 side, and thus the liquid flowing in from the feedback flow path 12 flows to the pump side and does not flow backward to the inlet 1 side. However, if necessary, a pinch valve or a nonreturn valve for preventing backflow may be provided in the flow path 3a at a point which is closer to the inlet 1 than the point where the feedback flow path 12 joins.

### Cell Dispersion Device Having Cell-Number Adjusting Function: Third Embodiment

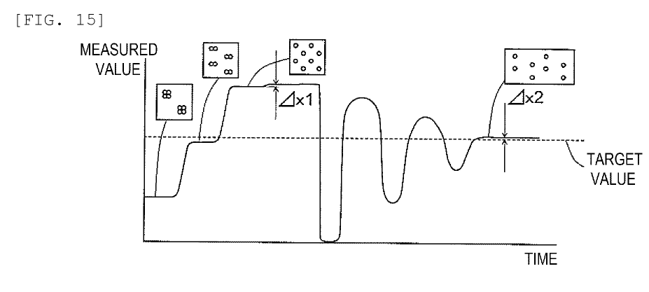

FIG. 13 is a schematic figure showing the third embodiment of the cell dispersion device having a cell-number adjusting function of the invention. The basic structure of the cell dispersion device 122 having a cell-number adjusting function according to the third embodiment is similar to that of the second embodiment, but a difference is that a buffer tank 14 is provided in the feedback flow path 12. The buffer tank 14 is as already explained in the description of the cell dispersion device 112 according to the third embodiment explained using FIG. 4.

The dilution step by the cell dispersion device 122 having a cell-number adjusting function according to the third embodiment is conducted as follows. First, the control unit 11 controls the switch valve 13 such that the flow path at the outlet 2 side is blocked and that the feedback flow path 12a side is selected and controls the switch valve 16 such that the feedback flow path 12b is blocked and that the flow path at the inlet 1 side is selected. In this state, the control unit 11 drives the peristaltic pump 4 and takes in the undiluted cell suspension from the inlet 1. The liquid taken in is delivered to the buffer tank 14. At this point, the undiluted cell suspension passes through the flow cell, and the light intensity is measured. The control unit 11 calculates the cell concentration from the results of the measurement, compares with the predetermined target value, calculates and determines the necessary amount of the diluent in view of the amount of the undiluted solution taken in and takes in the diluent from the diluent container 40 by switching the switch valve 49.

The undiluted cell suspension and the diluent taken in are mixed by circulating the cell suspension and the diluent in the circulation flow path composed of the feedback flow paths 12a and 12b including the buffer tank 14 and the flow paths 3a and 3b after switching the switch valve 16 such that the feedback flow path 12b is selected. The cell suspension having a desired cell concentration can be discharged from the outlet 2 by switching the switch valve 13 such that the flow path at the outlet 2 side is selected and then driving the peristaltic pump 4.

In the circulation flow path, the liquids which enter from the top drop in the buffer tank 14 and pass through the orifice 41, and the liquids are thus mixed. The capacity of the buffer tank 14 and the capacities of the flow paths can be appropriately determined in view of the efficiency of stirring in the buffer tank 14 and in the other flow paths, the expected liquid volume to be handled and the like. For example, when the liquid volume to be handled is in the range of about 120 mL to 180 mL, the capacity of the circulation flow path is 100 mL, and the capacity of the buffer tank 14 is 100 mL. When the buffer tank 14 is provided, an advantage that the measurement of the cell number becomes stable is also obtained because the air can be removed from the circulation flow path.

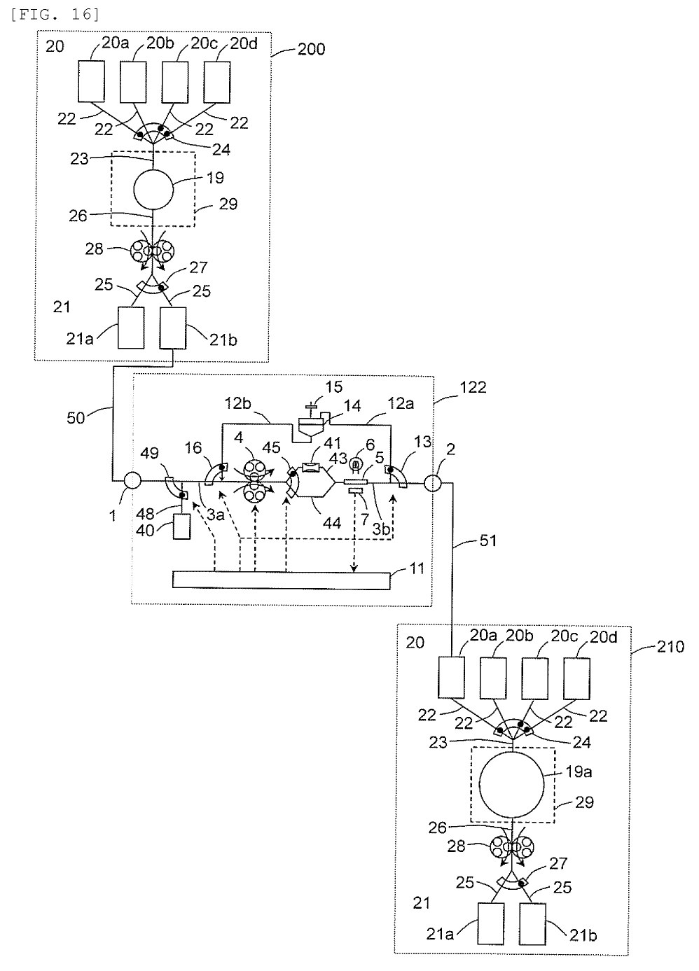

FIG. 14 is a schematic figure showing the cell dispersion device having a cell-number adjusting function according to a variation of the third embodiment. In the embodiments which have been explained above, the diluent is taken in by the peristaltic pump 4 which is also used for taking in the cell suspension and mixing the cell suspension and the diluent. A pump having a relatively large flow amount is efficient for taking in and mixing the suspension, but a pump having a large flow amount is not so suitable for fine adjustment for taking in the diluent. Also, the flow amount of the peristaltic pump 4 itself varies widely, and the peristaltic pump 4 is not so suitable for infusing a small amount of liquid. Thus, the cell dispersion device 123 having a cell-number adjusting function according to the variation shown in FIG. 14 is designed in such a manner that the diluent is added by a small-amount pump 46 which is newly provided. As the small-amount pump 46, for example, a diaphragm pump, a syringe pump and the like can be used. When the diluent is added by infusing the diluent to the buffer tank 14 in this variation, the switch valve 49 which is provided in the third embodiment is not necessary.

FIG. 15 is a conceptual figure showing the change with time in the light intensity value which is output from the detector 7 when a cell suspension containing cell aggregates is passed, through the cell dispersion device 122 having a cell-number adjusting function according to the third embodiment or the variation 123 thereof, subjected to the cell dispersion step for a while and then subjected to the cell-number adjustment step.

### Closed Subculture System Using Cell Dispersion Device Having Cell-Number Adjusting Function

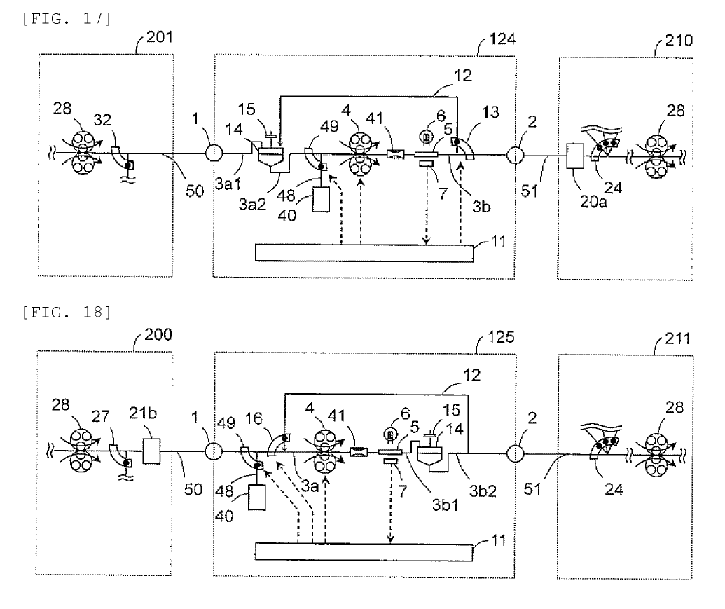

FIG. 16 is a schematic figure showing the whole structure of a subculture system using a cell dispersion device having a cell-number adjusting function. A closed subculture system can be formed by connecting the closed-system cell culture devices 200 and 210 which are similar to those of the subculture system described using FIG. 9 to the cell dispersion device 122 having a cell-number adjusting function through connecting flow paths 50 and 51, respectively. A cell dispersion device having a cell-number adjusting function having another structure which has been explained above may also be used.

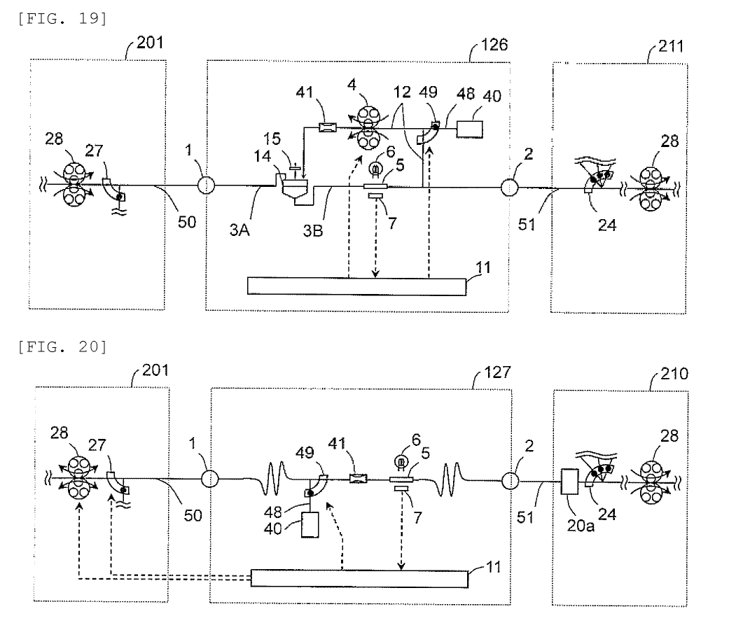

FIG. 17 is a schematic figure showing a part of the structure of the first variation of a subculture system using a cell dispersion device having a cell-number adjusting function. In this structure, the cell suspension discharged from a cell culture device for extended culturing is directly delivered to a cell dispersion device having a cell-number adjusting function. The basic structure of a cell culture device 201 is the same as that of the cell culture device 200, but the cell culture device 201 does not have a recovery bag for the cell suspension and is connected to a cell dispersion device 124 having a cell-number adjusting function through the connecting flow path 50. The cell dispersion device 124 having a cell-number adjusting function has a structure in which a buffer tank 14 is placed between a peristaltic pump 4 and an inlet 1. The flow path at the inlet 1 side and the flow path at the pump side are indicated by 3a1 and 3a2, respectively. 3a1 enters the buffer tank 14 from the top, and 3a2 exits from the bottom of the tank. A flow path 3b after the peristaltic pump 4 is branched, and one of the branched flow paths, a feedback flow path 12, is returned to the buffer tank 14 from the top. In this structure, a switch valve is not necessary at the point where the feedback flow path 12 joins. A switch valve 13 is provided at the branched part of the flow path 3b and the feedback flow path 12. A branch flow path 48 connected to a diluent container 40 is provided between the buffer tank 14 and the peristaltic pump 4. The cell dispersion solution discharged from the cell culture device 201 is delivered to the buffer tank 14 of the cell dispersion device 124 having a cell-number adjusting function by a peristaltic pump 28 of the cell culture device 201. After dispersing the cell aggregates and adjusting the cell concentration in the cell-number adjusting device 124, the cell suspension is delivered to a cell suspension supply bag 20a of the cell culture device 210 for subculturing through the flow path 51 because there is no buffer area in the flow path after the peristaltic pump 4 of the cell dispersion device 124 having a cell-number adjusting function.

FIG. 18 is a schematic figure showing a part of the structure of the second variation of a subculture system using a cell dispersion device having a cell-number adjusting function. In this structure, a cell culture device 211 for subculturing does not have a cell suspension supply bag. A cell dispersion device 125 having a cell-number adjusting function has a structure in which a buffer tank 14 is provided between a peristaltic pump 4 and an outlet 2. The flow path at the pump side and the flow path at the outlet 2 side are indicated by 3b1 and 3b2, respectively. 3b1 enters the buffer-tank 14 from the top, and 3b2 exits from the bottom of the buffer tank 14. The flow path 3b2 is branched, and one of the branched flow paths, a feedback flow path 12, is returned to a flow path 3a before the peristaltic pump. A switch valve 16 is provided at the point where the flow paths join. A branch flow path 48 connected to a diluent container 40 is provided in the flow path 3a between the buffer tank 14 and the peristaltic pump 4. In this structure, a switch valve is not necessary at the branched part of the flow path 3b2 and the feedback flow path 12. Because there is no buffer area between the flow path 3b2 and a peristaltic pump 28 of the culture device 211 for subculturing and there is no route for a liquid to escape, a liquid does not flow backward when the peristaltic pump 4 is driven even when there is no switch valve at the branched part to the feedback flow path 12. However, when the volume of air in this space is large, the air may expand, and thus a liquid may flow backward to the feedback flow path side from the outlet 2 due to the expanded air. Thus, a pinch valve or a nonreturn valve for preventing backflow may be provided between the branched part to the feedback flow path and the outlet 2 for preventing backflow. In this regard, the recovery bag 21b is provided in the cell culture device 200 for extended culturing and functions as a buffer area between the cell dispersion device 125 having a cell-number adjusting function. After dispersing the cell aggregates and adjusting the cell concentration in the cell dispersion device 125 having a cell-number adjusting function, the cell suspension is transported by driving the peristaltic pump 28 of the culture device 211 for subculturing and directly seeded in the culture device 211.

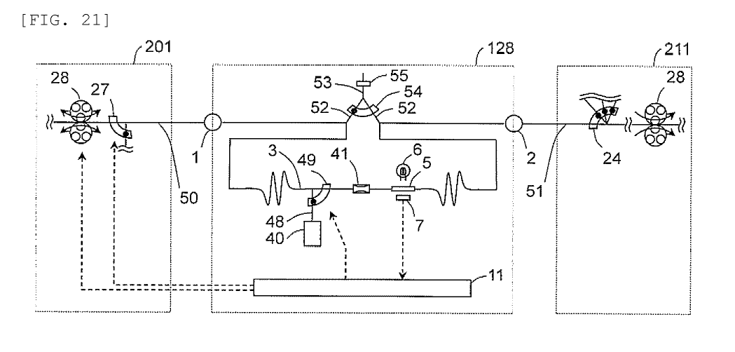

FIG. 19 is a schematic figure showing a part of the structure of the third variation of a subculture system using a cell dispersion device having a cell-number adjusting function. In this structure, none of the cell culture device for extended culturing, the cell dispersion device having a cell-number adjusting function and the cell culture device for subculturing has a buffer area. In the cell dispersion device 126 having a cell-number adjusting function, a buffer tank 14 is provided between flow paths 3A and 3B which connect an inlet 1 and an outlet 2, and a peristaltic pump 4 is provided in the middle of a feedback flow path 12. The feedback flow path 12 enters the buffer tank 14 from the top so that a switch valve is not necessary. A branch flow path 48 connected to a diluent container 40 is provided in the middle of the feedback flow path 12 and before the peristaltic pump 4. In this structure, the cell suspension delivered from the cell culture device 201 for extended culturing to the buffer tank 14 is circulated in the circulation flow path by driving the peristaltic pump 4. If necessary, a valve may be provided in the flow path 3A which connects the inlet 1 and the buffer tank 14 or in the flow path 3B after the branched part to the feedback flow path 12 and before the outlet 2. The cell suspension is delivered to the cell culture device 211 for extended culturing by driving a peristaltic pump 28 of the cell culture device 211.

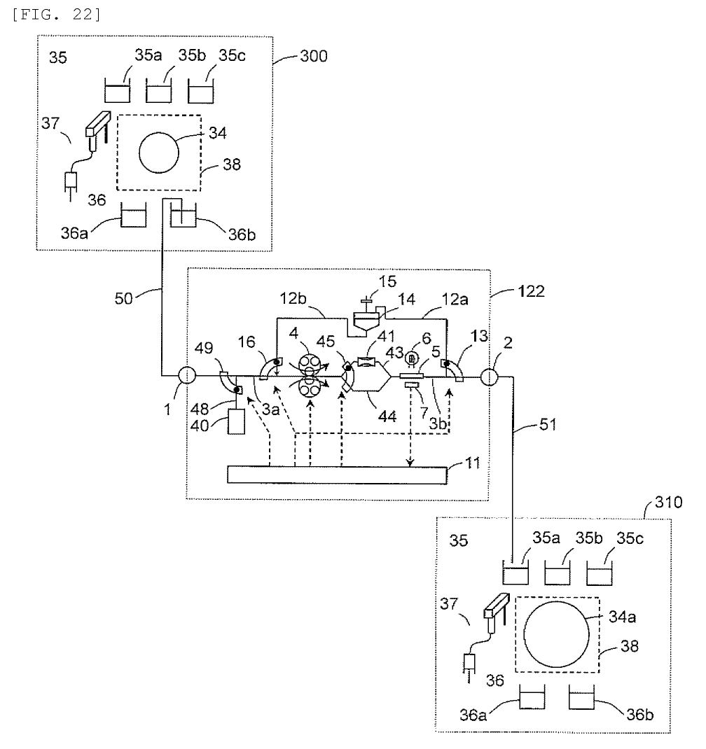

FIG. 20 is a schematic figure showing a part of the structure of the fourth variation of a subculture system using a cell dispersion device having a cell-number adjusting-function. The cell dispersion device 127 having a cell-number adjusting function used here does not have a feedback flow path and mixes liquids by causing the liquids to flow forward and backward. The cell dispersion device 127 having a cell-number adjusting function does not have its own peristaltic pump and causes a liquid to flow using a peristaltic pump of a culture device. The cell suspension is delivered to the cell dispersion device 127 having a cell-number adjusting function by driving the peristaltic pump 28 of the cell culture device 201 for extended culturing while a switch valve 27 of the culture device 201 selects the flow path 50. The diluent is taken in by switching a switch valve 49 such that the branch flow path side is opened and reversing the direction of rotation of the peristaltic pump 28. The liquids are mixed by returning the switch valve 49 and switching the direction of rotation of the peristaltic pump several times. After adjusting the cell concentration, the cell suspension is delivered to the supply bag 20a of the cell culture device 210 for subculturing by peristaltic pump of the cell culture device 201. Then, the connecting flow path 50 is closed by switching the switch valve 27 at the cell culture device 201 side.

FIG. 21 is a schematic figure showing a part of the structure of the fifth variation of a subculture system using a cell dispersion device having a cell-number adjusting-function. This structure is obtained by further modifying the fourth variation, and the cell suspension is received and sent between the cell culture devices and the cell dispersion device having a cell-number adjusting function directly. A cell dispersion device 128 having a cell-number adjusting function has a structure in which branch flow paths 52 are provided in a flow path 3 at the inlet 1 side and at the outlet 2 side and in which the two branch flow paths are connected to a common flow path 53 which is open to the atmosphere. The common flow path 53 can be switched with a switch valve 54, and a HEPA filter 55 is connected to the common flow path to prevent contamination by germs from outside. First, the switch valve 54 is switched such that the outlet 2 side is opened, and the switch valve 27 of the cell culture device 201 for extended culturing is switched such that the connecting flow path 50 is selected. When the peristaltic pump 28 is driven in this state, the cell suspension is delivered to the cell dispersion device 128 having a cell-number adjusting function. The light intensity is measured by a detector 7, and if necessary, the diluent is taken in by reversing the direction of rotation of the peristaltic pump 28 after switching a switch valve 49 such that the branch flow path side is opened. Then, the switch valve 27 at the cell culture device 201 side is switched, and the connecting flow path 50 is closed. After dispersing the cell aggregates and adjusting the cell concentration, the cell suspension is delivered to the cell culture device 211 by driving the peristaltic pump 28 of the cell culture device 211 after switching the switch valve 54 such that the branch flow path 52 at the outlet 2 side is blocked.

### Open Subculture System Using Cell Dispersion Device Having Cell-Number Adjusting Function

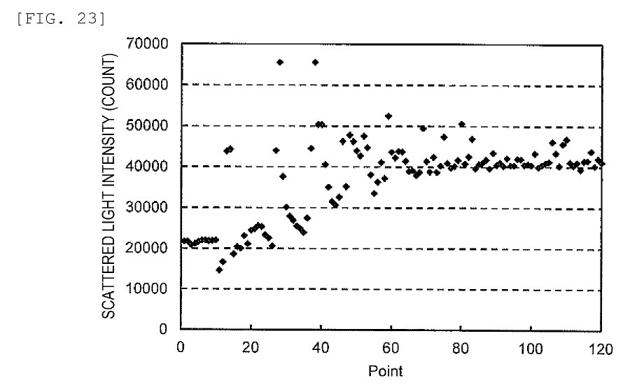

FIG. 22 is a schematic figure showing the whole structure of an open subculture system using a cell dispersion device having a cell-number adjusting function. As in the subculture system described using FIG. 10, the cell dispersion device having a cell-number adjusting function can also be connected to open-system cell culture devices and used.

### EXAMPLES

### (Cell Dispersion Test)

A total volume of a solution obtained by suspending 4.6×10⁶ cells of Caco-2 (a human colon cancer cell line) which had been cryopreserved at −80° C. in 9 mL of a culture medium for Caco-2 cells containing 10% FBS (Fetal Bovine Serum) was seeded and cultured in an extended culture container having a base area of 78.5 cm² of a cell culture device. As a result, the cells were at 80% confluence after two days. Then, the cells were washed with 3 mL of PBS and detached by adding 2 mL of 0.25% trypsin-1 mM EDTA and leaving the cells still at 37° C. for four minutes. The trypsin activity was stopped by adding 3 mL of the culture medium, and the culture medium containing the detached cells was recovered.

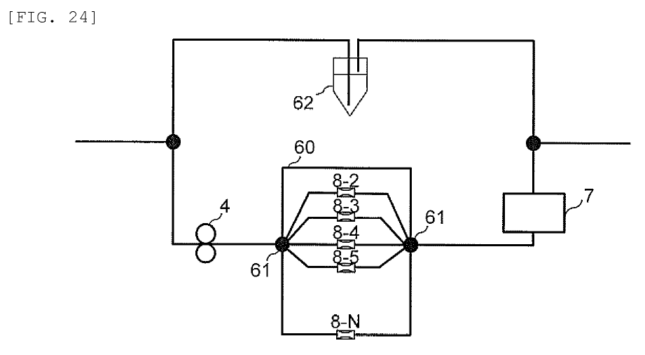

Five milliliters of the recovered culture medium containing the cells was supplied to a cell dispersion device having an equivalent structure to that shown in FIG. 3. The flow path of the cell dispersion device was composed of a silicone tube having an inside diameter of 3.15 mm, and the total length thereof was 520 mm. An orifice of inside diameter of 0.7 mm×length of 1 mm was provided in a part of the flow path. The flow cell tor measuring the light intensity used had a size of 5 mm. square. The scattered light intensity was measured (wavelength of 700 nm, measurement angle of 20°) intermittently by the detector while driving the peristaltic pump such that the sample circulated the circulation flow path about 10 times. As a result, a process in which the measured values converged with time was observed (FIG. 23). When, the state of the cell suspension was observed visually, it was determined that the cell dispersion state was equivalent to that obtained when cells were dispersed by pipetting the same sample manually about 10 times.

### (Automatic Optimization of Conditions for Dispersing Cells Using NIH/3T3 Cells)

An example of the automatic optimization of the conditions for dispersing cells is explained using the cell dispersion device shown in FIG. 24. In the device shown in FIG. 24, the flow path is circular. Parallel flow paths are provided in a part of the flow path, and a flow path 60 having no orifice and flow paths having orifices (8-2 to 8-N) having different inside diameters are provided in parallel. One of the parallel flow paths can be selected by switching multiway valves 61.

A solution obtained by recovering undispersed NIH/3T3 cells (3.0 to 4.0×10⁶ cells) with 20 mL of a medium was put into a sample reservoir 62, and the cell dispersion solution was circulated by driving a peristaltic pump 4. The conditions for dispersing were optimized by the following procedures.

First, the inside diameter and the length of the orifice were optimized. The flow path 60 having no orifice was composed of a flow path of 3.15 mm i.d.×720 mm as a whole. Orifices 8-2 to 8-7 having the following sizes were used: 1.6 mm i.d.×1 mm (8-2), 1.0 mm i.d.×1 mm (8-3), 0.7 mm i.d.×1 mm (8-4), 0.4 mm i.d.×1 mm (8-5), 0.4 mm i.d.×10 mm (8-6) and 0.4 mm i.d.×30 mm (8-7).

The flow rate and the driving period of the pump were fixed, and a detector 7 measured the cell suspension instrument which had passed through the flow path 60 having no orifice and sent the results to a control unit. A standard value had been set in the control unit in advance. When dispersion was determined to be insufficient, the multiway valves 61 were switched, and a new sample was passed through the next orifice 8-2. Similarly, the control unit determined the degree of dispersion of the cell sample which had passed through the orifice 8-2 and further passed the sample through 8-3, 8-4 and 8-5 in this order when the degree of dispersion was insufficient. This was continued until the results of the detector 7 cleared the standard value. When the standard value was not cleared after any of the conditions, the dispersion means which was the closest to the standard value was used for determining the optimum values. As a result, it was determined that the optimum inside diameter×optimum length of the orifice was 0.4 mm i.d.×30 mm.

Next, the flow rate was optimized. Under the conditions of a fixed orifice and a fixed driving period of the pump, the peristaltic pump 4 was regulated, and the flow rate to deliver the cell suspension was changed to 20 mL/min, 30 mL/min and 40 mL/min. The flow rate at which the dispersion degree was the closest to the standard value was used as the optimum value. As a result, it was determined that the optimum flow rate was 40 mL/min.

Next, the number of times the cells pass through the orifice was optimized. Because the number of times the cells pass through the orifice can be calculated from the flow rate and the driving period of the pump, the driving period of the pump was optimized. The orifice and the flow rate were fixed, and the degrees of dispersion after the passing periods of 90 seconds, 180 seconds and 270 seconds, which are in proportion to the number of times of passing, were evaluated. As a result, the optimum passing period was 180 seconds.

When NIH/3T3 cells were dispersed under the optimized conditions for dispersing and subcultured continuously, the viability after two days was 95% or more. In this regard, when the degree of dispersion does not clear the standard value even when treatment under the optimized conditions for dispersing is conducted, the same sample may be treated again under different conditions for dispersing. In this case, it is preferable to change the conditions in the following order: 1) reduce the inside diameter of the dispersion means; 2) increase the flow rate of the peristaltic pump; and 3) increase the length of the dispersion means.

All of the publications, the patents and the patent applications cited in this description are incorporated in this description as they are as reference.

### REFERENCE SIGNS LIST

1 . . . inlet, 2 . . . outlet, 3 . . . flow path, 4 . . . peristaltic pump, 5 . . . flow cell, 6 . . . light source, 7 . . . detector, 8 . . . orifice, 9 . . . flow-path pressing mechanism, 10 . . . switch valve, 11 . . . control unit, 12 . . . feedback flow path, 13 . . . switch valve, 14 . . . buffer tank, 15 . . . HEPA filter, 16 . . . switch valve, 19 . . . culture container, 20 . . . supply bag, 21 . . . recovery bag, 22 . . . individual flow path, 23 . . . common flow path, 24 . . . switch valve, 25 . . . individual flow path, 26 . . . common flow path, 27 . . . switch valve, 28 . . . peristaltic pump, 34 . . . culture container, 35 . . . supplied solution container, 36 . . . recovered solution container, 37 . . . dispensing mechanism, 38 . . . incubator, 40 . . . diluent container, 41 . . . orifice, 43 . . . flow path, 44 . . . flow path, 45 . . . switch valve, 46 . . . small-amount pump, 47 . . . diluent flow path, 48 . . . branch flow path, 49 . . . switch valve, 50 . . . connecting flow path, 51 . . . connecting flow path, 52 . . . branch flow path, 53 . . . common flow path, 54 . . . switch valve, 55 . . . HEPA filter, 60 . . . flow path having no orifice, 61 . . . other-way valve, 62 . . . sample reservoir, 102 . . . cell-number adjusting device, 110-114 . . . cell dispersion device, 120-128 . . . cell dispersion device having cell-number adjusting function, 200 and 210 . . . cell culture device (closed system), 300 and 310 . . . cell culture device (open system)

1. A cell-suspension processing device for dispersing cell aggregates included in a cell suspension,
having an inlet for taking in the cell suspension, an outlet for discharging the processed cell suspension and a flow path which is provided between the inlet and the outlet and which is capable of holding the cell suspension,
wherein the flow path has, provided thereto, a liquid delivery pump for causing the cell suspension inside to flow, a cell-dispersion-degree measurement instrument for measuring the dispersion degree of cells in the cell suspension and a narrow part for imparting shearing force to the cell suspension flowing inside,
the cell-suspension processing device has a control unit for controlling at least the liquid delivery pump on the basis of light intensity values obtained by the cell-dispersion-degree measurement instrument having a light source and a detector, and
the control unit determines whether the cells have attained a prescribed dispersion degree on the basis of a change with time in the light intensity values converging on a certain value obtained by the cell-dispersion-degree measurement instrument and, in a case when the cells have not attained the prescribed dispersion degree, drives the liquid delivery pump such that the cell suspension is passed through the narrow part.

2. The cell-suspension processing device according to claim 1, wherein the narrow part is provided by a flow-path pressing mechanism which presses a flow path made of an elastic material and which sets the degree of narrowness of the flow path at any degree, and the control unit controls the flow-path pressing mechanism on the basis of the data obtained by the cell-dispersion-degree measurement instrument.

3. The cell-suspension processing device according to claim 1, wherein the flow path has a parallel flow path part in which at least two or more flow paths are provided in parallel and which is designed such that a part of the flow paths is selected with a switch valve to pass the cell suspension, and the narrow part is provided in at least one of the flow paths included in the parallel flow path part.

4. The cell-suspension processing device according to claim 3, wherein narrow parts are provided in two or more of the flow paths included in the parallel flow path part, and the sectional areas of the narrow parts are different.

5. The cell-suspension processing device according to claim 3, wherein the control unit is capable of controlling the switch valve, and the control unit controls the switch valve such that any of the flow paths in the parallel flow path part is selected on the basis of the data obtained by the cell-dispersion-degree measurement instrument.

6. The cell-suspension processing device according to claim 1, wherein the cell-dispersion-degree measurement instrument measures the intensity of the scattered light or the transmitted light of light applied to the cell suspension and collects data on the dispersion degree of cells as a light intensity value, and the control unit determines the degree of dispersion of the cell aggregates on the basis of the change with time in the light intensity value.

7. An automatic subculture system including a first cell culture device for extended culturing, a cell-suspension processing device for dispersing cell aggregates included in a cell suspension and a second cell culture device for subculturing,
wherein the cell-suspension processing device has an inlet for taking in a cell suspension discharged from the first cell culture device, an outlet for discharging the processed cell suspension and a flow path which is provided between the inlet and the outlet and which is capable of holding the cell suspension,
the flow path has, provided thereto, a liquid delivery pump for causing the cell suspension inside to flow, a cell-dispersion-degree measurement instrument for measuring the dispersion degree of cells in the cell suspension and a narrow part for imparting shearing force to the cell suspension flowing inside,
the cell-suspension processing device has a control unit for controlling at least the liquid delivery pump on the basis of light intensity values obtained by the cell-dispersion-degree measurement instrument having a light source and a detector, and
the control unit determines whether the cells have attained a prescribed dispersion degree on the basis of a change with time in the light intensity values converging on a certain value obtained by the cell-dispersion-degree measurement instrument and, in a case when the cells have not attained the prescribed dispersion degree, drives the liquid delivery pump such that the cell suspension is passed through the narrow part.
